(19) United States
(12) Patent Application Publication
(10) Pub. No.: US 2025/0017467 A1
(43) Pub. Date: Jan. 16, 2025

(54) WEARABLE AUTONOMOUS NETWORKING COOPERATION TERMINAL, SYSTEM THEREOF AND COMMUNICATION METHOD THEREOF

(71) Applicant: Hebei Qianxing Technology Co., Ltd., Shijiazhuang City (CN)

(72) Inventors: Xuhui Liu, Shenzhen City (CN); Xingzhi Huang, Nanping City (CN)

(21) Appl. No.: 18/238,634

(22) Filed: Aug. 28, 2023

(30) Foreign Application Priority Data
Jul. 11, 2023 (CN) 202310847838.3
Jul. 11, 2023 (CN) 202321816796.9

(51) Int. Cl. A61B 5/00

(52) U.S. Cl. CPC A61B 5/0024; A61B 5/6802; A61B 5/749

(57) ABSTRACT

The present disclosure provides a wearable autonomous networking cooperation terminal, a system thereof and a communication method thereof. A terminal device includes a voice communication device and a vital sign acquisition device. The voice communication device includes a main control module, a DECT communication module, a voice codec module, a speaker, a microphone, a TDMA/TDD processing module, a GFSK modulation module, a radio frequency module, a first wireless communication module and an antenna. The vital sign acquisition device includes a microcontroller, a second wireless communication module, a positioning module, a heart rate sensor, a body temperature sensor, a blood oxygen sensor, an acceleration sensor and a touch screen. The present disclosure realizes high-quality and low-delay voice service, and sharing and cooperative transmission of personal vital signs and position coordinates of a wearer of the terminal device, and improves the data interaction capability of the device.

### CROSS REFERENCE TO RELATED APPLICATION

This application claims takes priority from and claims the benefit of Chinese Patent Application No. 202310847838.3 filed on Jul. 11, 2023, and Chinese Patent Application No. 202321816796.9 filed on Jul. 11, 2023, the contents of which are herein incorporated by reference.

### TECHNICAL FIELD

The present disclosure belongs to the technical field of smart wearable devices, and in particular, relates to a wearable autonomous networking cooperation terminal, a system thereof and a communication method thereof.

### BACKGROUND

There are many types of smart wearable products, such as devices that can be put on or worn, contact with a human body, acquire human health information, assist users in listening to or seeing content, and transmit data with smart phones, all of which can be collectively referred to as “smart wearable devices”. Smart wearable devices are in close contact with the human body, which can acquire a variety of human data and provide interactive experiences such as vision, touch, hearing and health monitoring.

The connection of smart wearable devices is an indispensable key step for interaction and control among various devices. However, at present, the interconnection communication between smart wearable devices usually uses Bluetooth, WIFI and a mobile network for networking communication, but there are corresponding defects in networking communication in outdoor scenes. However, Bluetooth and WIFI are both short-distance communication, and are not suitable for long-distance communication. Although the mobile network can realize long-distance networking instant communication, there are problems of channel conflict and network signal crosstalk. In the process of instant communication, each networking personnel cannot view and obtain individual vital sign data from each other in real time. The data interaction capability is limited.

### SUMMARY

Therefore, the wearable autonomous networking cooperation terminal, a system thereof and a communication method thereof according to the present disclosure are helpful to solve the problems that it is difficult for the existing wearable devices to realize high-quality long-distance networking communication and the data interaction capability is poor.

In order to achieve the above purpose, the present disclosure uses the following technical scheme.

In a first aspect, the present disclosure provides a wearable autonomous networking cooperation terminal, comprising: a voice communication device and a vital sign acquisition device; wherein
the voice communication device comprises a main control module, a DECT communication module, a voice codec module, a speaker, a microphone, a TDMA/TDD processing module, a GFSK modulation module, a radio frequency module, a first wireless communication module and an antenna; one end of each of the speaker, the microphone, the DECT communication module, the voice codec module, the TDMA/TDD processing module, the GFSK modulation module, the first wireless communication module and the radio frequency module is connected with the main control module, and the other end of the radio frequency module is connected with the antenna;
the vital sign acquisition device comprises a microcontroller, a second wireless communication module, a positioning module, a heart rate sensor, a body temperature sensor, a blood oxygen sensor, an acceleration sensor and a touch screen; one end of each of the second wireless communication module, the positioning module, the heart rate sensor, the body temperature sensor, the blood oxygen sensor, the acceleration sensor and the touch screen is connected with the microcontroller;
the first wireless communication module and the second wireless communication module are wirelessly connected through wireless signals.

Further, both the first wireless communication module and the second wireless communication module comprise a BLE Bluetooth module.

Further, the radio frequency module comprises a radio frequency chip; the radio frequency chip has a frequency range of 1880 MHZ-1900 MHZ.

Further, the positioning module comprises a Beidou positioning module and a GPS positioning module.

Further, the voice communication device further comprises: a voice input signal amplification circuit and a voice output signal amplification circuit; an input end of the voice input signal amplification circuit is connected with the microphone, an output end of the voice input signal amplification circuit is connected with the main control module, an input end of the voice output signal amplification circuit is connected with the main control module, and an output end of the voice output signal amplification circuit is connected with the speaker.

In a second aspect, the present disclosure provides a communication system of a wearable autonomous networking cooperation terminal, comprising:
at least two wearable autonomous networking cooperation terminals according to the first aspect; wherein the wearable autonomous networking cooperation terminal is autonomously connected to a DCET network channel through a voice communication device working in a frequency range to form a DECT networking.

Further, the DECT networking comprises a master and slavers, and all of the slavers are connected with the master for bidirectional communication.

In a third aspect, the present disclosure provides a communication method of a wearable autonomous networking cooperation terminal, which is suitable for a master in the communication system and comprises:
acquiring, by a master, personal position coordinates and current vital sign data of a master user;
receiving, by the master, personal position coordinates and current vital sign data uploaded by all slavers in a DECT networking;
sharing, by the master, personal position coordinates and current vital sign data of all of the slavers and the master to the DECT networking.

In a fourth aspect, the present disclosure provides a communication method of a wearable autonomous networking cooperation terminal, which is suitable for a slaver in the communication system and comprises:
acquiring, by a slaver, personal position coordinates and current vital sign data of a slaver user, and uploading the personal position coordinates and the current vital sign data to a master through a DECT networking;
receiving, by the slaver, personal position coordinates and current vital sign data of the master and other slavers in the DECT networking, and displaying the position coordinates and the vital sign data of the master and other slaver users in the DECT networking.

By using the technical scheme, the present disclosure at least has the following beneficial effects.

The wearable autonomous networking cooperation terminal according to the present disclosure specifically comprises a voice communication device and a vital sign acquisition device. The voice communication device comprises a main control module, a DECT communication module, a voice codec module, a speaker, a microphone, a TDMA/TDD processing module, a GFSK modulation module, a radio frequency module, a first wireless communication module and an antenna. The vital sign acquisition device comprises a microcontroller, a second wireless communication module, a positioning module, a heart rate sensor, a body temperature sensor, a blood oxygen sensor, an acceleration sensor and a touch screen. The first wireless communication module of the voice communication device and the second wireless communication module of the vital sign acquisition device establish wireless communication connection through wireless signals. In the present disclosure, the personal position coordinates and the current vital sign data of the wearer are acquired by the vital sign acquiring device, and the acquired data are transmitted to the voice communication device by wireless transmission. Then, the voice communication device is used for long-distance DECT networking. The personal position coordinates and the current vital sign data of the wearer are shared and transmitted to other voice communication devices in the DECT networking. After other wearers receive the personal position coordinates and the vital sign data of other wearers in the DECT networking through the voice communication devices, the personal position coordinates and the vital sign data are transmitted to the vital sign acquisition device for display. While ensuring the terminal device to achieve high-quality and low-delay voice service, sharing and cooperative transmission of personal vital signs and position coordinates of a wearer is realized, and the data interaction capability of the terminal device is improved.

It is to be understood that both the foregoing general description and the following detailed description are exemplary and illustrative only, rather than limit the present disclosure.

### BRIEF DESCRIPTION OF THE DRAWINGS

In order to explain the technical scheme in the embodiment of the present disclosure or the prior art more clearly, the drawings needed in the description of the embodiment or the prior art will be briefly introduced hereinafter.

FIG. 1 is a schematic structural diagram of a wearable autonomous networking cooperation terminal according to an exemplary embodiment.

FIG. 2 is a schematic diagram of a circuit structure of a voice communication device according to an exemplary embodiment.

FIG. 3 is a schematic diagram of a circuit structure of a vital sign acquisition device according to an exemplary embodiment.

FIG. 4 is a schematic structural diagram of dial function keys of a Bluetooth watch according to an exemplary embodiment.

FIG. 5 is an architecture diagram of a terminal device networking communication according to an exemplary embodiment.

FIG. 6 is an architecture diagram of a communication system of a wearable autonomous networking cooperation terminal according to an exemplary embodiment.

FIG. 7 is a schematic diagram of a DECT network networking of a plurality of terminal devices according to an exemplary embodiment.

FIG. 8 is a flowchart of a communication method of a master according to an exemplary embodiment.

FIG. 9 is a flowchart of a communication method of a slaver according to an exemplary embodiment.

### DETAILED DESCRIPTION OF THE EMBODIMENTS

In order to make the purpose, the technical scheme and the advantages of the present disclosure more clear, the technical scheme of the present disclosure will be described in detail hereinafter.

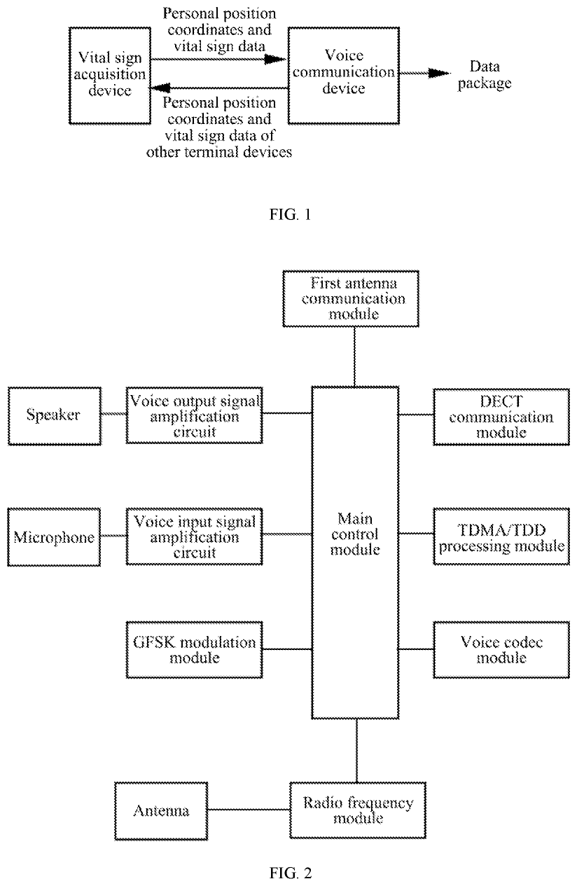

As shown in FIG. 1, FIG. 1 is a wearable autonomous networking cooperation terminal according to an exemplary embodiment. The terminal device comprises a voice communication device and a vital sign acquisition device.

As shown in FIG. 2, the voice communication device comprises a main control module, a DECT communication module, a voice codec module, a speaker, a microphone, a TDMA/TDD processing module, a GFSK modulation module, a radio frequency module, a first wireless communication module and an antenna; one end of each of the speaker, the microphone, the DECT communication module, the voice codec module, the TDMA/TDD processing module, the GFSK modulation module, the first wireless communication module and the radio frequency module is connected with the main control module, and the other end of the radio frequency module is connected with the antenna;

The DECT communication module is configured to transmit and receive communication data. The voice codec module is configured to encode the acquired voice signal into a digital signal or decode the received digital signal into a voice signal. The GFSK modulation module is configured to modulate the digital signals into digital radio frequency signals or demodulate the digital radio frequency signals into the digital signals. The TDMA/TDD processing module is configured to perform time division multiple access decomposition of the demodulated digital radio frequency signals or perform channel codec interleaving and encryption of the digital radio frequency signals to be issued to form a time division multiple access data stream. The radio frequency module is configured to transmit the digital radio frequency signals to the antenna, so as to output or receive the digital radio frequency signals transmitted by the antenna. The first wireless communication module is configured to receive personal vital sign data and personal position coordinates of the user acquired by the vital sign acquiring device, and transmit, to the vital sign acquiring device, corresponding operation instructions, shared terminal data, and so on.

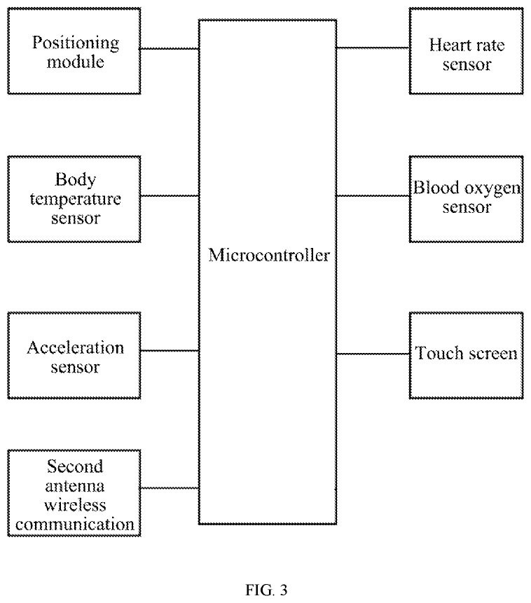

As shown in FIG. 3, the vital sign acquisition device comprises a microcontroller, a second wireless communication module, a positioning module, a heart rate sensor, a body temperature sensor, a blood oxygen sensor, an acceleration sensor and a touch screen; one end of each of the second wireless communication module, the positioning module, the heart rate sensor. the body temperature sensor, the blood oxygen sensor, the acceleration sensor and the touch screen is connected with the microcontroller;

The second wireless communication module is configured to transmit the personal vital sign data and personal position coordinates of the user which are acquired to the voice communication device, and to receive and display the shared terminal data transmitted by the voice communication device. The positioning module is configured to acquire the personal position coordinate data of the end user. The heart rate sensor, the body temperature sensor and the blood oxygen sensor are used to acquire the current vital sign data of the end user, including heart rate, body temperature and blood oxygen. The acceleration sensor is configured to acquire the current displacement of the end user. The touch screen is used for the end user to view the vital sign data and the received shared terminal data.

Specifically, the first wireless communication module and the second wireless communication module are wirelessly connected through wireless signals to establish wireless communication.

Specifically, the voice communication device of the present disclosure carries out long-distance autonomous networking cooperation communication based on the DECT wireless network, so as to adapt to outdoor networking application scenarios. The DECT is a new generation of public digital cordless telephone standard first formulated by the European Telecommunications Standards Organization in 1992, which has the following characteristics: 1. providing high-quality and low-delay voice service; 2. supporting data communication service; 3. supporting automatic lossless switching, including carrier and time slot switching resulted from the deterioration of channel characteristics in the cell; 4. using the dynamic channel allocation technology without complicated frequency planning; 5. low power consumption; 6. a wireless carrier frequency band: 1880 MHZ-1900 MHZ; 7. number of carriers: 10; 8. the center frequency of the carrier is 1897.344-C*1.728 MHZ (c=0, 1 . . . 9); 9. the bandwidth of 1.728 MHz; 10. the modulation mode: GFSK, BT=0.5; 11. the access mode: TDMA/TDD; 12. the information rate: 1152 Kbps; 13. the net channel rate: 6.4Kbps in A domain for control and signaling transmission. and 32 Kbps in B domain for voice service and data transmission; 14. voice encoding: 32 Kbps ADPCM.

In one embodiment, both the first wireless communication module and the second wireless communication module in the present disclosure comprise a BLE Bluetooth module. that is, wireless communication is established between the voice communication device and the vital sign acquisition device through Bluetooth signals to transmit data. In addition, other wireless communication units can also be used to establish wireless communication, such as an RTU remote terminal unit, a DTU module, an LORA module, a ZigBee wireless communication module, etc., which can be selected according to actual application needs.

Further, in one embodiment, the radio frequency module comprises a radio frequency chip. As the voice communication device of the present disclosure uses the DECT wireless network for networking. Therefore, the radio frequency chip in the present disclosure has a frequency range of 1880 MHZ-1900 MHZ.

Further, in one embodiment, the positioning module specifically comprises a Beidou positioning module and a GPS positioning module. Through the Beidou positioning module and the GPS positioning module, the geographical position of the end user is positioned, and the corresponding personal position coordinates are obtained.

Further, in one embodiment, the voice communication device further comprises: a voice input signal amplification circuit and a voice output signal amplification circuit; an input end of the voice input signal amplification circuit is connected with the microphone, an output end of the voice input signal amplification circuit is connected with the main control module, an input end of the voice output signal amplification circuit is connected with the main control module. and an output end of the voice output signal amplification circuit is connected with the speaker.

Specifically, when a voice signal is issued, the voice signal is input via the microphone and enters the main control module through the voice input signal amplification circuit. Similarly. when receiving the voice signal, the voice signal is finally output to the speaker through the voice output signal amplification circuit.

In the specific practice, the voice communication device of the present disclosure can also be improved based on the existing device. For example, the existing headset based on the DECT is improved, a BLE Bluetooth module is added to the headset, and the BLE Bluetooth module is electrically connected with the main control unit of the headset to realize wireless communication and data transmission with the vital sign acquisition device. The headset based on the DECT can be realized by referring to the existing patent document with the patent application number of CN 209472616 U, which is not described in detail here.

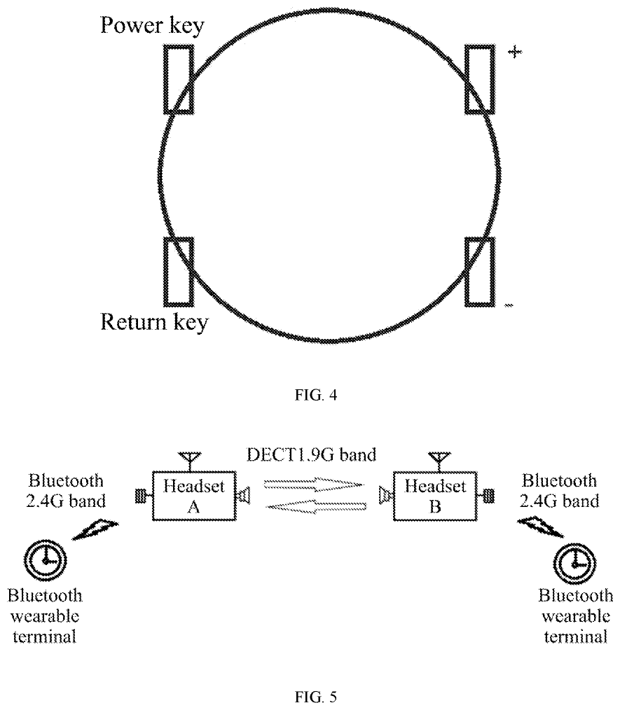

In the specific practice, the vital sign acquisition device of the present disclosure can be realized by using the existing Bluetooth wearable terminals with health monitoring and positioning, for example. Bluetooth watches of models such as Irbon WACH MAX-A90, OPPO watch3 Pro. HUAWEI WATCH 3 Pro, etc. In addition, the vital sign acquisition device can also be improved based on the existing Bluetooth watch. Specifically, the vital sign acquisition device according to the present disclosure is designed in the form a Bluetooth wearable terminal. As shown in FIG. 4, the dial keys of the Bluetooth wearable terminal designed in the present disclosure comprise: a power key, which is used to turn on and off the device, and can turn on and off the device after being pressed for 1 second; a return key, which is pressed on the main dial interface to directly enter the dial switching interface, in which other interfaces are to return to the previous menu; a selecting key+, which is used to switch the next menu interface and is pressed for 1 second to 2 seconds after selection for confirmation; and a selecting key−, which is used to switch the previous menu interface and is pressed for 1 second to 2 seconds after selection for confirmation. The circuit structure inside the Bluetooth wearable terminal is realized by referring to the above vital sign acquisition device, and these four function keys are connected with the circuit structure inside the Bluetooth wearable terminal, so as to obtain the final Bluetooth wearable terminal

Specifically, in one embodiment, the present disclosure uses two wearable autonomous networking cooperation terminals for networking communication. The Bluetooth wearable terminal is used as the vital sign acquisition device, and the DECT headset is used as the voice communication device. As shown in FIG. 5, the communication process between the wearable autonomous networking cooperation terminals is specifically as follows.

The daily use form of the Bluetooth wearable terminal is a personal wearable device. which carries out data interaction with the BLE Bluetooth module in headset A through Bluetooth 2.4 GHZ signals. Headset A transmits personal vital signs and position coordinate information monitoring data through headset B in the network with 6.4 Kbps bandwidth of DECT A domain working at 1.9 GHZ, including but not limited to: device ID. GPS/Beidou coordinates. heart rate chart, electrocardiogram, blood oxygen, blood pressure, etc.

Further, in an alternative embodiment, according to the present disclosure, the voice communication device is integrated into the vital sign acquisition device to form an integral terminal device, and the BLE Bluetooth communication module is not added. At this time, the wearable autonomous networking cooperation terminal comprises: a main control module, a DECT communication module, a voice codec module, a speaker, a microphone, a TDMA/TDD processing module, a GFSK modulation module, a radio frequency module, an antenna, a positioning module, a heart rate sensor, a body temperature sensor, a blood oxygen sensor, an acceleration sensor and a touch screen. The main control module is connected with the DECT communication module, the voice codec module, the speaker, the microphone, the TDMA/TDD processing module, the GFSK modulation module, the radio frequency module, the positioning module, the heart rate sensor, the body temperature sensor, the blood oxygen sensor, the acceleration sensor and the touch screen, respectively; the radio frequency module is connected with the antenna.

At this time, the specific working principle of the wearable autonomous networking cooperation terminal is as follows. First, the terminal device is connected to the DECT network for networking. At the same time, the user wears the wearable autonomous networking cooperation terminal. The vital sign data of the user is acquired through the heart rate sensor, the body temperature sensor and the blood oxygen sensor, and the position coordinates are acquired through the positioning module. The main control module transmits the vital sign data and position coordinates which are acquired to other terminal device in the form of digital radio frequency signals through the radio frequency module. The other terminal device analyzes and displays the received digital radio frequency signals to obtain the vital sign data and the position coordinates of the user. When the distance of all the terminal devices is below 500 meters, the display is in normal map scale, when it's below 1 km, 2 km, the map scales will change accordingly.

In addition, when making a voice communication, the user transmits and receives data through the DECT communication module in the wearable autonomous networking cooperation terminal. Then, the acquired voice signals are encoded into digital signals by using the voice codec module, or the received digital signals are decoded into voice signals, so as to make a voice communication with other terminal devices in the DECT networking.

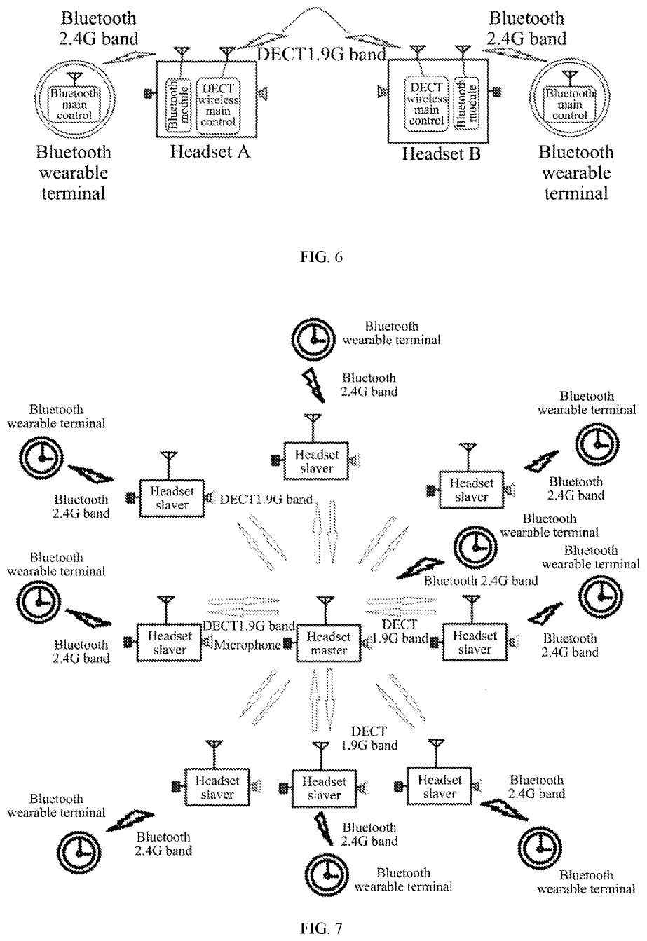

As shown in FIG. 6, the present disclosure further provides a wearable autonomous networking cooperation system, comprising:
at least two wearable autonomous networking cooperation terminals described above; wherein the wearable autonomous networking cooperation terminal is autonomously connected to a DCET network channel through a voice communication device working in a frequency range to form a DECT networking.

Specifically, the DECT networking comprises a master and slavers, and all of the slavers are connected with the master for bidirectional communication.

The master transmits data to the slaver in a Write mode; and the slaver transmits data to the master in a Notify mode.

Specifically, in an alternative embodiment, the wearable autonomous networking cooperation terminal of the present disclosure uses a Bluetooth wearable terminal and a DECT headset, both of which carry out wireless communication based on the BLE Bluetooth module. In the networking communication process, the communication process between the Bluetooth wearable terminal and the DECT headset is as follows.

1. the description of BLE Profile is set, specifically including:
Service UUID: 0xF000EAE0-0000-4000-0000-00000000B000;
Feature (TX) UUID: 0xF000EAE1-0451-4000-0000-00000000B000, attribute: Write;
Feature (RX) UUID: 0xF000EAE2-0451-4000-0000-00000000B000, attribute: Notify.

The master transmits data to the slaver, and the length of the transmitted data is set to 20 bytes in the Write mode.

The slaver transmits data to the master, and the length of the transmitted data is set to 20 bytes in the Notify mode.

BLE communication only uses these two features UUID for interaction. Refer to the next procedure for the specific protocol format.

In the present disclosure, “the Bluetooth wearable terminal” is used as a master and can be used as a slaver in an OTA mode to upgrade in the air. The “DECT headset” is used as a slaver.

### 2. BLE Protocol

A) The master transmits a 20-byte data packet to the slaver, and then the slaver returns a 20-byte data packet. All data packets have a fixed length of 20 bytes.

B) The format of the 20-byte data packet transmitted by the master is as follows:

| B0 | B1 | B2 - - - B19 |
|---|---|---|
| header | command | payload bytes, 18 bytes |

Header: fixed as 0xFC, command: the value range of 0x00→0x7F, in which the most significant bit of the command is reserved as 0.

C) A 20-byte response packet is returned to the master after the slaver receives the command with the following format.

| | |
|---|---|
| B0 | B1 - - - B19 |

| | | |
|---|---|---|
| Bit[7] | Bit[6-0] | |
| status | command | payload bytes, 19 bytes |

### 3. BLE Data Format

(1) the Bluetooth wearable terminal transmits information to the headset.

Command format: 0xFC 00 A1 A2 B1 B2 B3 B4 C1 C2 C3 C4 D1 D2 D3 D4 Hr 00 00 00.

Function: the Bluetooth wearable terminal transmits its own information (location, heart rate) to the headset with a total of 20 bytes.

Description: A1A2, which is the device ID, is used to identify the device or personnel. B1B2B3B4, which is the UTC timestamp, represents the time point when this piece of data is generated. C1C2C3C4 is a longitude, and D1D2D3D4 is a dimension, which need to be restored and analyzed as float data. Hr is the heart rate value (0-255).

Command reply: after receiving and finishing the analysis, the headset returns the status to the master according to the analysis result.

Correct execution: 0x00 00 00 00 00 00 00 00 00 00 00 00 00 00 00 00 00 00 00 00;

Failed execution: 0x80 00 00 00 00 00 00 00 00 00 00 00 00 00 00 00 00 00 00 00

(2) The headset transmits information to the Bluetooth wearable terminal.

Command format: 0xFC 01 A1 A2 B1 B2 B3 B4 Cl C2 C3 C4 D1 D2 D3 D4 Hr 00 00 00.

Function: the headset forwards information (location, heart rate) of other GPS devices (other Bluetooth wearable terminals) to the Bluetooth wearable terminal with a total of 20 bytes. Every time the location information of one device is transmitted, the Bluetooth wearable terminal distinguishes devices according to ID (A1A2) and updates the coordinate points on the display screen.

Description: A1A2, which is the device ID, is used to identify the device or personnel. B1B2B3B4, which is the UTC timestamp, represents the time point when this piece of data is generated. C1C2C3C4 is a longitude, and D1D2D3D4 is a dimension, which need to be restored and analyzed as float data. Hr is the heart rate value (0-255).

Command reply: because the headset transmits data to the Bluetooth wearable terminal in a continuous notify mode, the master does not reply. If the master receives the data successfully, the screen display will be automatically updated.

In addition, the data update frequency of the Bluetooth wearable terminal can also be set, that is, how often the Bluetooth wearable terminal transmits data to the headset. At present, according to the present disclosure, the Bluetooth wearable terminal transmits data to the headset every 1 second.

Specifically, based on the communication process between the Bluetooth wearable terminal and the DECT headset, the present disclosure uses nine wearable autonomous networking cooperation terminals for networking communication. Specifically, the DECT networking form is shown in FIG. 7. The headset is connected by a master to eight slavers. Nine headsets are connected to nine Bluetooth wearable terminals through Bluetooth 2.4G signals, respectively. The Bluetooth wearable terminal is attached to the user. Personal position coordinates and vital sign data are acquired through the Bluetooth wearable terminal, and the information of nine Bluetooth wearable terminals in the network is gathered at the master in the headset and is issued to nine Bluetooth wearable terminals for display, so that any Ethernet Bluetooth terminal which is networked can view the personal position coordinates and the current vital sign data of nine other people in the network.

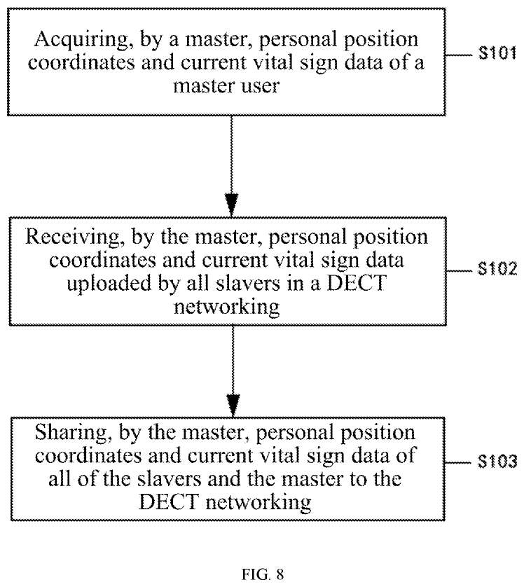

As shown in FIG. 8, the present disclosure further provides a communication method of a wearable autonomous networking cooperation terminal, which is suitable for the master in the above embodiment and comprises:
S101: acquiring, by a master, personal position coordinates and current vital sign data of a master user;
S102: receiving, by the master, personal position coordinates and current vital sign data uploaded by all slavers in a DECT networking;
S103: sharing, by the master, personal position coordinates and current vital sign data of all of the slavers and the master to the DECT networking.

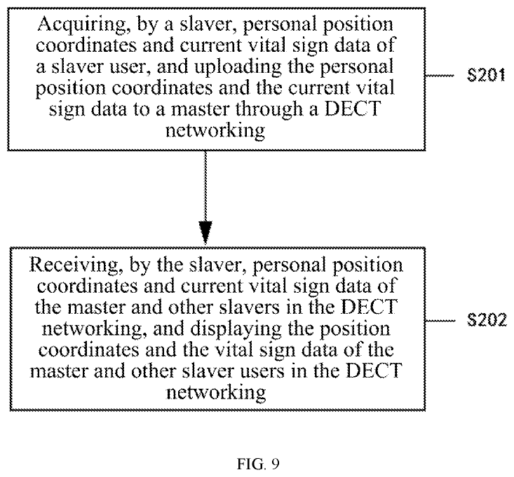

As shown in FIG. 9, the present disclosure further provides a communication method of a wearable autonomous networking cooperation terminal, which is suitable for the slaver in the above embodiment and comprises:
S201: acquiring, by a slaver, personal position coordinates and current vital sign data of a slaver user, and uploading the personal position coordinates and the current vital sign data to a master through a DECT networking;
S202: receiving, by the slaver, personal position coordinates and current vital sign data of the master and other slavers in the DECT networking, and displaying the position coordinates and the vital sign data of the master and other slaver users in the DECT networking.

In the present disclosure, the personal position coordinates and the current vital sign data of the wearer are acquired by the vital sign acquiring device, and the acquired data are transmitted to the voice communication device by wireless transmission. Then, the voice communication device is used for long-distance DECT networking. The personal position coordinates and the current vital sign data of the wearer are shared and transmitted to other voice communication devices in the DECT networking. After other wearers receive the personal position coordinates and the vital sign data of other wearers in the DECT networking through the voice communication devices, the personal position coordinates and the vital sign data are transmitted to the vital sign acquisition device for display. While ensuring the terminal device to achieve high-quality and low-delay voice service, sharing and cooperative transmission of personal vital signs and position coordinates of a wearer is realized, and the data interaction capability of the terminal device is improved.

It can be understood that the same or similar parts in the above embodiments can refer to each other. What is not explained in detail in some embodiments can refer to the same or similar parts in other embodiments.

In the description of this specification, descriptions referring to the terms “one embodiment”, “some embodiments”, “examples”, “specific examples” or “some examples” mean that specific features, structures, materials or characteristics described in connection with the embodiment or example are included in at least one embodiment or example of the present disclosure. Although the embodiments of the present disclosure have been shown and described above, it can be understood that the above embodiments are exemplary and cannot be understood as limitations of the present disclosure. Those skilled in the art can make changes, modifications, substitutions and variations to the above embodiments within the scope of the present disclosure.

What is claimed is:

1. A wearable autonomous networking cooperation terminal, comprising: a voice communication device and a vital sign acquisition device; wherein
the voice communication device comprises a main control module, a DECT communication module, a voice codec module, a speaker, a microphone, a TDMA/TDD processing module, a GFSK modulation module, a radio frequency module, a first wireless communication module and an antenna; one end of each of the speaker, the microphone, the DECT communication module, the voice codec module, the TDMA/TDD processing module, the GFSK modulation module, the first wireless communication module and the radio frequency module is connected with the main control module, and the other end of the radio frequency module is connected with the antenna;
the vital sign acquisition device comprises a microcontroller, a second wireless communication module, a positioning module, a heart rate sensor, a body temperature sensor, a blood oxygen sensor, an acceleration sensor and a touch screen; one end of each of the second wireless communication module, the positioning module, the heart rate sensor, the body temperature sensor, the blood oxygen sensor, the acceleration sensor and the touch screen is connected with the microcontroller;
the first wireless communication module and the second wireless communication module are wirelessly connected through wireless signals.

2. The wearable autonomous networking cooperation terminal according to claim 1, wherein both the first wireless communication module and the second wireless communication module comprise a BLE Bluetooth module.

3. The wearable autonomous networking cooperation terminal according to claim 1, wherein the radio frequency module comprises a radio frequency chip; the radio frequency chip has a frequency range of 1880 MHz-1900 MHz.

4. The wearable autonomous networking cooperation terminal according to claim 1, wherein the positioning module comprises a Beidou positioning module and a GPS positioning module.

5. The wearable autonomous networking cooperation terminal according to claim 1, wherein the voice communication device further comprises: a voice input signal amplification circuit and a voice output signal amplification circuit; an input end of the voice input signal amplification circuit is connected with the microphone, an output end of the voice input signal amplification circuit is connected with the main control module, an input end of the voice output signal amplification circuit is connected with the main control module, and an output end of the voice output signal amplification circuit is connected with the speaker.

6. A communication system of a wearable autonomous networking cooperation terminal, comprising:
at least two wearable autonomous networking cooperation terminals according to claim 1; wherein the wearable autonomous networking cooperation terminal is autonomously connected to a DCET network channel through a voice communication device working in a frequency range to form a DECT networking.

7. The communication system of the wearable autonomous networking cooperation terminal according to claim 6, wherein the DECT networking comprises a master and slavers, and all of the slavers are connected with the master for bidirectional communication.

8. A communication method of a wearable autonomous networking cooperation terminal, comprising:
acquiring, by a master, personal position coordinates and current vital sign data of a master user;
receiving, by the master, personal position coordinates and current vital sign data uploaded by all slavers in a DECT networking;
sharing, by the master, personal position coordinates and current vital sign data of all of the slavers and the master to the DECT networking.

9. A communication method of a wearable autonomous networking cooperation terminal, comprising:
acquiring, by a slaver, personal position coordinates and current vital sign data of a slaver user, and uploading the personal position coordinates and the current vital sign data to a master through a DECT networking;
receiving, by the slaver, personal position coordinates and current vital sign data of the master and other slavers in the DECT networking, and displaying the position coordinates and the vital sign data of the master and other slaver users in the DECT networking.
